(19) United States
(12) Patent Application Publication
(10) Pub. No.: US 2021/0118539 A1
(43) Pub. Date: Apr. 22, 2021

(54) MEDICATION MANAGEMENT DEVICE, MEDICATION MANAGEMENT METHOD, AND NON-TRANSITORY COMPUTER-READABLE STORAGE MEDIUM STORING MEDICATION MANAGEMENT PROGRAM

(71) Applicant: OMRON HEALTHCARE Co., Ltd., Kyoto (JP)

(72) Inventors: Hironori Sato, Kyoto (JP); Fumihiko Nakamura, Kyoto (JP); Daisuke Nozaki, Kyoto (JP)

(21) Appl. No.: 17/101,867

(22) Filed: Nov. 23, 2020

Related U.S. Application Data
(63) Continuation of application No. PCT/JP2019/020056, filed on May 21, 2019.

(30) Foreign Application Priority Data
May 29, 2018 (JP) 2018-102327

(51) Int. Cl. G16H 20/10; G16H 10/60; G16H 50/20

(52) U.S. Cl. CPC G16H 20/10; G16H 50/20; G16H 10/60

(57) ABSTRACT

A medication management device includes an acquisition unit configured to acquire medication information by medicine and dosing information by medicine related to a managed subject, a setting unit configured to set at least one unit medication period, a calculation unit configured to calculate a dosing adherence rate by medicine for each of the at least one unit medication periods on the basis of the medication information by medicine and the dosing information by medicine thus acquired and the at least one unit medication period thus set, and a generation unit configured to generate management screen data configured to display, in association with each other, the medication information by medicine thus acquired and the dosing adherence rate by medicine thus calculated for each of the at least one unit medication periods.

### CROSS-REFERENCE TO RELATED APPLICATIONS

This application is the U.S. national stage application filed pursuant to 35 U.S.C. 365(c) and 120 as a continuation of International Patent Application No. PCT/JP2019/020056, filed May 21, 2019, which application claims priority from Japanese Patent Application No. 2018-102327, filed May 29, 2018, which applications are incorporated herein by reference in their entireties.

### TECHNICAL FIELD

This invention relates to a medication management device, a medication management method, and a non-transitory computer-readable storage medium storing a medication management program configured to manage medication in a treatment of a managed subject having a disease that requires medication at home, at work, or the like, for example.

### BACKGROUND ART

Patent Document 1 discloses a treatment support device configured to manage medication in a clinical trial. This treatment support device manages the medication of a managed subject by acquiring biological information transmitted from the subject.

### CITATION LIST

### Patent Literature

Patent Document 1: JP 6040112 B

### SUMMARY OF INVENTION

### Technical Problem

In the treatment support device of Patent Document 1, the presence or absence of the onset of side effects is determined on the basis of the biological information transmitted from the managed subject. Then, an efficacy of the medication is determined on the basis of the onset status of side effects. In such a medication management method, when a medicine is changed, it is difficult to identify which medicine is less likely to be taken by the managed subject, or which medicine is likely to be missed by the managed subject.

The present invention has been made with reference to the circumstances described above, and it is an object of the present invention to provide a medication management device, a medication management method, and a non-transitory computer-readable storage medium storing a medication management program that make it possible to easily identify, by medicine, a dosing status of a managed subject, for example, thereby enabling accurate medication management.

### Solution to Problem

In order to solve the problems described above, the present invention adopts the following measures, for example.

That is, a medication management device according to an example of the present disclosure includes an acquisition unit configured to acquire medication information by medicine and dosing information by medicine related to a managed subject, a setting unit configured to set at least one unit medication period, a calculation unit configured to calculate a dosing adherence rate by medicine for each of the at least one unit medication periods on the basis of the medication information by medicine and the dosing information by medicine thus acquired and the at least one unit medication period thus set, and a generation unit configured to generate management screen data configured to display, in association with each other, the medication information by medicine thus acquired and the dosing adherence rate by medicine thus calculated for each of the at least one unit medication periods.

According to the configuration described above, a medication administrator, such as a physician or a pharmacist, is able to confirm, by management screen data, medication information by medicine and a dosing adherence rate by medicine for each unit medication period in a treatment for a managed subject having a disease that requires medication management at home or at work. For example, the medication administrator can compare, on a dosing adherence rate management screen, the results of dosing in response to medication instructions on a per unit medication period basis and by medicine, thereby easily determining what kind of medication does not reduce adherence (enthusiasm for treatment) for the managed subject, that is, which medicine is easy to take and less likely to be missed for the managed subject.

The medication management device according to the example described above is configured to manage medication in a treatment for a managed subject having a hypertension disorder.

In the medication management device according to the example described above, as the medication information, medicine identification information, a prescribed dosage by medicine, and a medication start date and a medication end date by medicine are acquired.

In the medication management device according to the example described above, the acquisition unit is configured to acquire, as the dosing information, at least medicine identification information and a dosing date by medicine.

Here, the acquisition unit is configured to determine, by medicine, whether the managed subject has taken the medicine on the basis of the dosing information.

In the medication management device according to the example described above, the acquisition unit is configured to further acquire a medication start date and a medication end date, and the setting unit is configured to set the medication start date as a start date of the at least one unit medication period, and set the medication end date as an end date of the at least one unit medication period.

Here, the acquisition unit is configured to acquire a first hospital visit date as the medication start date, and acquire a second hospital visit date corresponding to a hospital visit date following the first hospital visit date as the medication end date.

In the medication management device according to the example described above, the calculation unit is configured to calculate, as the dosing adherence rate by medicine, a ratio of an actual number of doses to a prescribed number of doses for each of the medicines in the at least one unit medication period.

In the medication management device according to the example described above, the acquisition unit is configured to acquire, as the dosing information, at least a dosing date and a dosing time by medicine, and the generation unit is configured to set a plurality of prescribed dosing times in the at least one unit medication period and generate, on the basis of the dosing date and the dosing time by medicine thus acquired, management screen data configured to display, in association with each other, the medication information by medicine and the dosing adherence rate by medicine for each of the plurality of prescribed dosing times.

Here, in a case where there are a plurality of types of medicines to be taken, medication identification information is further acquired as the dosing information.

### Advantageous Effects of Invention

According to the present invention, it is possible to provide a medication management device, a medication management method, and a non-transitory computer-readable storage medium storing a medication management program that make it possible to easily identify, in the treatment of a disease that requires medication management at home, which medicine is easy to take and less likely to be missed for a managed subject, such as a patient.

### BRIEF DESCRIPTION OF DRAWINGS

FIG. 1 is a block diagram illustrating a functional configuration of a medication management device according to an application example.

FIG. 2 is a schematic diagram illustrating a configuration of a medication management system including the medication management device according to a first embodiment.

FIG. 3 is a block diagram illustrating a hardware configuration of a blood pressure measurement device according to the first embodiment.

FIG. 4 is a block diagram illustrating a hardware configuration of a portable terminal according to the first embodiment.

FIG. 5 is a block diagram illustrating a hardware configuration of a physician terminal according to the first embodiment.

FIG. 6 is a block diagram illustrating a hardware configuration of a server according to the first embodiment.

FIG. 7 is a block diagram illustrating a functional configuration of a medication management circuit as an example of the medication management device according to the first embodiment.

FIG. 8 is a flowchart illustrating a processing procedure for medication management by a medication management system including the medication management circuit as an example of the medication management device according to the first embodiment.

FIG. 9 is a flowchart illustrating a procedure of a side effect management process of the medication management circuit serving as the example of the medication management device according to the first embodiment.

FIG. 10 is a flowchart illustrating a procedure of a dosing adherence rate management process of the medication management circuit serving as an example of the medication management device according to the first embodiment.

FIG. 11 is a drawing illustrating a side effect management screen displayed on the basis of output screen data generated by a side effect management process of the medication management circuit serving as an example of the medication management device according to the first embodiment.

FIG. 12 is a drawing illustrating a dosing adherence rate management screen displayed on the basis of output screen data generated by a dosing adherence rate management process of the medication management circuit serving as an example of the medication management device according to the first embodiment.

### DESCRIPTION OF EMBODIMENTS

Now, with reference to the drawings, embodiments are described. Note that, in the following description, constituent elements having the same function and configuration are denoted with a shared reference symbol. Further, when a plurality of constituent elements having a shared reference symbol are distinguished from one another, distinguishment is made by adding additional symbols following the shared reference symbol. Note that, when there is no particular need in distinguishing a plurality of constituent components, the plurality of constituent components are denoted only with a shared reference symbol without an additional symbol.

### 1. Application Example

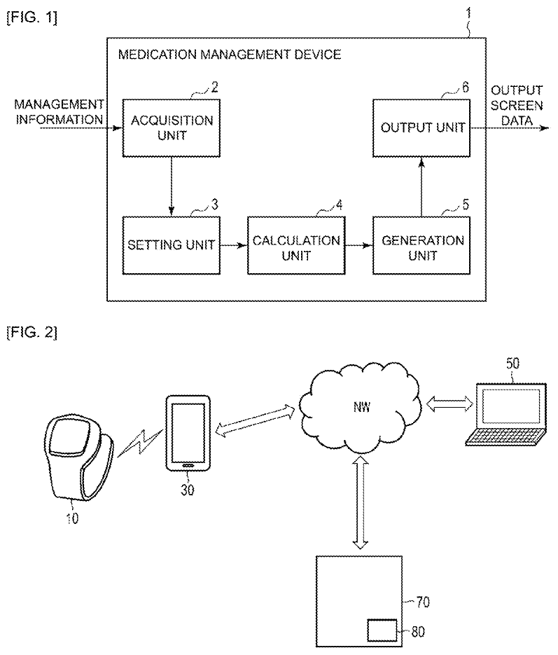

First, with reference to FIG. 1, an example of a medication management device to which the present invention is applied will be described.

A medication management device 1 is used for medication management by an administrator in a treatment of a managed subject having a disease that requires medication management at home. The administrator is, for example, a physician. The managed subject is, for example, a patient having a target disease. The medication management device 1 executes a diagnosis screen generation process on the basis of a diagnosis screen generation program. The diagnosis screen generation program is an example of a medication management program. As illustrated in FIG. 1, the medication management device 1 includes an acquisition unit 2, a setting unit 3, a calculation unit 4, a generation unit 5, and an output unit 6.

The acquisition unit 2 acquires management information related to the managed subject. The management information includes medication information and dosing information. The acquisition unit 2 transmits the management information thus acquired to the setting unit 3.

The medication information is information related to specified medicines of the medication. The medication information includes, for each of the specified medicines of the medication, a medication start date, a medication end date, medicine identification information, and a prescribed dosage, for example. The medicine identification information may include a medicine name.

The dosing information is information related to actual dosing of the managed subject. The dosing information includes at least the medicine identification information and a dosing date/time for each medicine. The dosing information can include, in addition to the dosing date/time, a dosage and the like, by medicine. The medicine identification information is, for example, a medicine name.

The setting unit 3 sets at least one unit medication period on the basis of the medication information, for example. A start date of the unit medication period is, for example, consistent with the medication start date, and an end date of the unit medication period is, for example, consistent with the medication end date. The medication start date and the medication end date may be included in the dosing information, for example. In each of the unit medication periods, the medicine identification information (medicine name, for example) and the prescribed dosage specified in the medication are the same. The unit medication period may be one day or may be a number of days. Further, lengths of the unit medication periods may differ from each other. The setting unit transmits the setting information of the unit medication period to the calculation unit 4.

The calculation unit 4 calculates the dosing adherence rate for each unit medication period and for each medicine on the basis of the medication information, the dosing information, and the setting information of the unit medication period. The dosing adherence rate of a specific medicine in a specific unit medication period is a ratio of the number of times that the specific medicine was actually taken to the prescribed number of doses of the specific medicine within the specific unit medication period. The calculation unit 4 transmits the dosing adherence rate thus calculated to the generation unit 5.

The generation unit 5 executes a dosing adherence rate management screen generation process, thereby generating dosing adherence rate management screen data for the managed subject on the basis of the medication information, the setting information of the unit medication period, and the dosing adherence rate calculated by the calculation unit 4. The dosing adherence rate management screen data are display data for displaying the dosing adherence rate management screen on a display screen of a display device. On the dosing adherence rate management screen, the medication information and the dosing adherence rate are arranged in correspondence with each unit medication period and in correspondence with each medicine, for example. The generation unit 5 transmits the dosing adherence rate management screen data thus generated to the output unit 6 as output screen data.

The output unit 6 outputs the dosing adherence rate management screen data generated by the generation unit 5 as output screen data to a display device or the like.

With a configuration such as described above, in a treatment for a diagnosed subject having a disease that requires medication management at home, the dosing adherence rate management screen that displays the medication information and the dosing adherence rate related to the managed subject for each unit medication period and for each medicine can be displayed on a display screen of a display device utilized by the administrator. By comparing the information on the dosing adherence rate management screen for each unit medication period, the administrator can easily determine the medication that does not lower adherence for the managed subject. Further, by comparing the information on the dosing adherence rate management screen for each medicine, the administrator can easily determine the medicine that does not lower adherence for the managed subject. Therefore, in the treatment of a disease that requires medication management at home, such as a hypertension disorder, an administrator such as a physician can easily identify which medicine is easy to take and less likely to be missed for a managed subject, such as a patient.

### 2. First Embodiment

A first embodiment of the medication management device according to the application example described above is described below. The following describes a medication management system that includes a server provided with a medication management circuit. The medication management circuit is an example of a medication management device.

### 2.1 Overall Configuration Example

FIG. 2 is a drawing schematically illustrating an example of an application scenario of the medication management system according to the present embodiment. The medication management system according to the present embodiment is a system for managing the medication of a managed subject having a hypertension disorder. Hypertension disorder is an example of a lifestyle-related disease. A lifestyle-related disease is also an example of a disease that requires medication management at home.

In the example of FIG. 2, the medication management system includes a physician terminal 50 and a server 70. The physician terminal 50 and the server 70 can be connected via a network NW, such as the Internet. A plurality of the physician terminals 50 may be provided. For communication between the physician terminal 50 and the server 70, near-field wireless communication or wired communication may be applied without going through the network NW. In the example of FIG. 2, the medication management system further includes a blood pressure measurement device 10 and a portable terminal 30. A plurality of each of the blood pressure measurement device 10 and the portable terminal 30 may be provided. In this case, the blood pressure measurement device 10 and the portable terminal 30 are connected by near-field wireless communication or wired communication. The portable terminal 30 can be connected to the server 70 via the network NW. The portable terminal 30 may be further connected to the physician terminal 50 via the network NW. Thus, the blood pressure measurement device 10 can be connected to the server 70 (and the physician terminal 50) via the portable terminal 30. That is, the blood pressure measurement device 10 is capable of communicating with the server 70 (and the physician terminal 50) via the portable terminal 30.

For example, the blood pressure measurement device 10 is a wearable device that can be worn at a desired measurement location (for example, a wrist). The blood pressure measurement device 10 measures a blood pressure value of the managed subject at the measurement location. The blood pressure measurement device 10 transmits the blood pressure information including the measurement result of the blood pressure value and the like to the portable terminal 30. Further, the blood pressure measurement device 10 can acquire the pulse information of the managed subject. The pulse information includes a pulse value and a pulse wave. The blood pressure measurement device 10 can transmit the pulse information to the portable terminal 30. The blood pressure measurement device 10 includes a clock function and can transmit the blood pressure information and the pulse information to the portable terminal 30 in association with the measured date/time.

The portable terminal 30 is, for example, a terminal that can be carried by the managed subject. The portable terminal 30 receives the blood pressure information and the pulse information from the blood pressure measurement device 10. The portable terminal 30 can save the received blood pressure information and pulse information along with the measured date/time of the blood pressure information and the pulse information, for example. Further, the portable terminal 30 can transfer the saved blood pressure information and pulse information in association with the measured date/time to the server 70 as appropriate. For example, the blood pressure information can include representative information such as a first measurement result (systolic blood pressure value and diastolic blood pressure value) in the morning after waking up, and an average value of the measurement results. Further, with the portable terminal 30, the dosing information and the side effect information of the managed subject can be input. The portable terminal 30 can transfer the dosing information and the side effect information to the server 70.

The physician terminal 50 is a terminal operable by an administrator, such as a physician. The administrator, such as a physician, consults with the managed subject and diagnoses a medical condition of the managed subject on the basis of test data and the like, for example. The physician terminal 50 can receive test data from a test device (not illustrated) or the like in a hospital, and present the test data to the administrator. From the physician terminal 50, the diagnosis information related to the managed subject is input by an operation by the administrator. Further, in the physician terminal 50, the medication information of the managed subject is input by an operation by the administrator. The physician terminal 50 can transmit the diagnosis information and the medication information to the server 70.

The server 70 is a server computer that accumulates information transmitted from the portable terminal 30, the physician terminal 50, and the like. The accumulated information is stored as an electronic medical chart, for example.

The server 70 includes a medication management circuit 80. The medication management circuit 80 acquires the management information related to the managed subject, and generates management screen data related to the managed subject on the basis of the management information thus acquired. The management screen data include side effect management screen data, dosing adherence rate management screen data, and the like. The server 70 can transmit the management screen data thus generated as output data to the physician terminal 50. Note that the server 70 may generate a portion of the management screen data as simplified screen data, and transmit the data to the portable terminal 30 as output data for the portable terminal 30. Further, the medication management circuit 80 is an example of a medication management device. A portion or all of the medication management circuit 80 may be provided to, for example, the physician terminal 50 or the like.

The physician terminal 50 can receive the output data generated by the medication management circuit 80 and display a side effect management screen and a dosing adherence rate management screen on the basis of the received output data. The physician terminal 50 is an example of a display device.

### 2.2 Hardware Configuration Examples

An example of a hardware configuration of each device of the medication management system according to the present embodiment will now be described.

### 2.2.1 Hardware Configuration Example of Blood Pressure Measurement Device

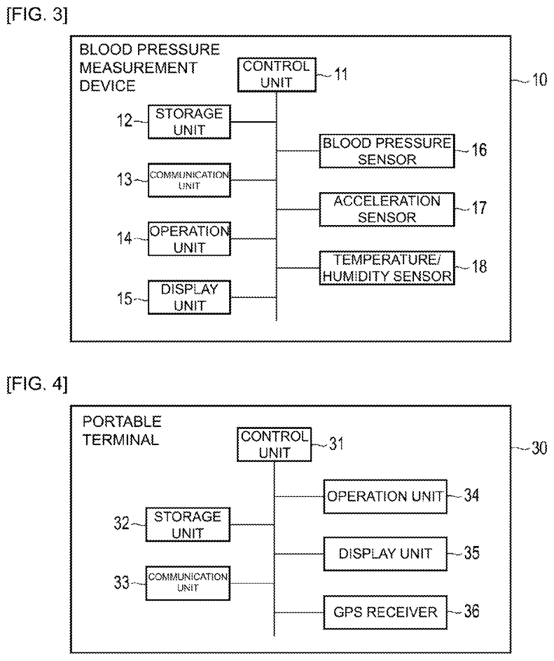

First, a hardware configuration example of the blood pressure measurement device 10 according to the present embodiment will be described. FIG. 3 is a block diagram illustrating an example of the hardware configuration of the blood pressure measurement device 10 according to the present embodiment. As illustrated in FIG. 3, the blood pressure measurement device 10 according to the present embodiment includes a control unit 11, a storage unit 12, a communication unit 13, an operation unit 14, a display unit 15, and a blood pressure sensor 16. The blood pressure measurement device 10 may further include at least one of an acceleration sensor 17 and a temperature/humidity sensor 18.

The control unit 11 includes a central processing unit (CPU), a random-access memory (RAM), a read-only memory (ROM), and the like, and controls each component in accordance with information processing. Further, the control unit 11 includes a clock (not illustrated), and has a function of acquiring a current date/time. The control unit 11 may have a function of displaying the acquired date/time on the display unit 15.

The control unit 11 generates blood pressure information, pulse information, activity information, and environment information on the basis of measurement results from the blood pressure sensor 16, the acceleration sensor 17, and the temperature/humidity sensor 18. The blood pressure information and the pulse information include, for example, the measurement results of the blood pressure value and the pulse value as well as the pulse wave of the managed subject from the blood pressure sensor 16, and the like. The activity information includes an activity amount, the number of steps, and a sleep condition of the managed subject based on measurements from the acceleration sensor 17. The environment information includes a temperature and a humidity in the periphery of the managed subject based on measurements from the temperature/humidity sensor 18. Each of the blood pressure information, the pulse information, the activity information, and the environment information is associated with the measured date/time based on the current date/time acquired by the clock. Further, each of the blood pressure information, the pulse information, the activity information, and the environment information may further be associated with a device ID for uniquely identifying the blood pressure measurement device 10.

The storage unit 12 is, for example, an auxiliary storage device such as a solid state drive, for example. In a case where the blood pressure measurement device 10 is configured as a somewhat large device rather than a small device such as a watch type, the storage unit 12 may be a hard disk drive. The storage unit 12 stores programs executed by the control unit 11, the blood pressure information, the pulse information, the activity information, the environment information, and the like.

The communication unit 13 is a communication interface for performing communication with the portable terminal 30. The communication unit 13 transmits, to the portable terminal 30, the blood pressure information, the pulse information, the activity information, the environment information, and the like, for example. In the present embodiment, for communication with the portable terminal 30 by the communication unit 13, near-field wireless communication such as Bluetooth (trade name), for example, can be applied, but the communication is not limited thereto. For example, communication performed by the communication unit 13 may adopt communication via the network NW such as a local area network (LAN) or wired communication through use of a communication cable.

For example, the operation unit 14 includes a user interface such as a touch panel and an operation button. The operation unit 14 detects an operation performed by the managed subject via the user interface, and outputs a signal indicating a content of the operation to the control unit 11.

The display unit 15 includes, for example, a display screen (for example, a liquid crystal display (LCD), an electroluminescent (EL) display, or the like), an indicator, and the like. The display unit 15 displays information in accordance with a signal from the control unit 11, and notifies the managed subject of the information. The display unit 15 can display, for example, the blood pressure information, the pulse information, the activity information, the environment information, and the like stored in the storage unit 12.

The blood pressure sensor 16 measures a blood pressure value of the managed subject. The blood pressure value includes representative indices such as, for example, a systolic blood pressure and a diastolic blood pressure.

The blood pressure sensor 16 may be, for example, a continuous measurement type capable of measuring the blood pressure of the managed subject per heartbeat (continuously), or may be a non-continuous measurement type capable of measuring the blood pressure on the spot at a predetermined time (non-continuously). For example, the continuous measurement type blood pressure sensor 16 may adopt a method of measuring the blood pressure of the managed subject continuously on the basis of pulse transit time (PTT), a method of measuring the blood pressure of the managed subject continuously on the basis of a pressure pulse wave (tonometry method), or the like. Note that the method of measuring blood pressure continuously is not limited to the above-mentioned examples, and a method of detecting a pulse wave through use of a light emitting element and the like may be adopted as appropriate. For example, the non-continuous measurement type blood pressure sensor 16 may adopt a method of detecting a pulse wave by applying a pressure on a blood vessel through use of a cuff as a pressure sensor (oscillometric method). Thus, the blood pressure sensor 16 is capable of acquiring the pulse information.

The acceleration sensor 17 detects acceleration of the managed subject that occurs at the worn location of the blood pressure measurement device 10, as a group of three-axial components. Further, the acceleration sensor 17 may further includes a gyro sensor, and may further detect an angular velocity as a group of three-axial components in addition to acceleration.

The temperature/humidity sensor 18 measures a temperature and a humidity in the periphery of the managed subject.

### 2.2.2 Hardware Configuration Example of Portable Terminal

Next, a hardware configuration example of the portable terminal 30 is described. FIG. 4 is a block diagram illustrating an example of the hardware configuration of the portable terminal 30 according to the present embodiment. As illustrated in FIG. 4, the portable terminal 30 according to the present embodiment includes a control unit 31, a storage unit 32, a communication unit 33, an operation unit 34, a display unit 35, and a global positioning system (GPS) receiver 36.

The control unit 31 and the storage unit 32 are similar to the control unit 11 and the storage unit 12 of the blood pressure measurement device 10, respectively. The storage unit 32 of the portable terminal 30, under the control of the control unit 31, stores the information received from the blood pressure measurement device 10 and position information generated by the GPS receiver 36. The information received from the blood pressure measurement device 10 includes the blood pressure information, the pulse information, the activity information, the environment information, and the like. Further, the storage unit 32, under the control of the control unit 31, stores the dosing information, the side effect information, and the like input at the operation unit 34. Note that, when the blood pressure information, the pulse information, the activity information, the environment information, the dosing information, the side effect information, and the like are stored in the storage unit 32, the information can be stored together with an acquired date/time of the information.

The communication unit 33 is a communication interface for performing communication with the blood pressure measurement device 10 and the server 70 (and the physician terminal 50). The communication unit 33 receives, for example, the blood pressure information, the pulse information, the activity information, the environment information, and the like from the blood pressure measurement device 10. Further, the communication unit 33 transmits the blood pressure information, the pulse information, the activity information, the environment information, the position information, and the like to the server 70. Further, the communication unit 33 can also receive simple management screen data and the like from the server 70.

The operation unit 34 and the display unit 35 are similar to the operation unit 14 and the display unit 15 of the blood pressure measurement device 10, respectively. The display unit 35 can display an input screen of the dosing information and the side effect information. At the operation unit 34, the dosing information and the side effect information are input. For example, as the side effect information, feel good, dry cough, headache, dizziness, palpitation, hot flash, swelling, and the like can be input.

The GPS receiver 36 measures a position of the portable terminal 30, and generates the position information. For example, the position information includes a positioning date/time and a latitude and a longitude of the portable terminal 30 at the positioning date/time. For example, positioning performed by the GPS receiver 36 may be performed in synchronization with measurement performed by the blood pressure sensor 16 of the blood pressure measurement device 10.

### 2.2.3 Hardware Configuration Example of Physician Terminal

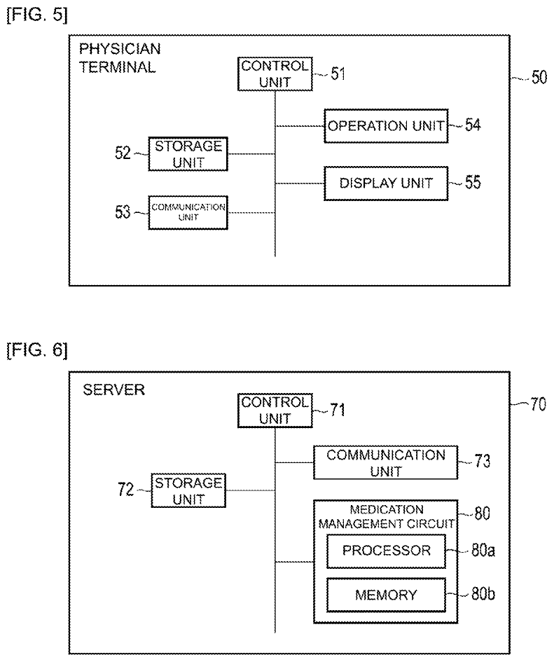

Next, a hardware configuration example of the physician terminal 50 is described. FIG. 5 is a block diagram illustrating an example of the hardware configuration of the physician terminal 50 according to the present embodiment. As illustrated in FIG. 5, the physician terminal 50 according to the present embodiment includes a control unit 51, a storage unit 52, a communication unit 53, an operation unit 54, and a display unit 55.

The control unit 51 and the storage unit 52 are similar to the control unit 11 and the storage unit 12 of the blood pressure measurement device 10, respectively. The control unit 51 of the physician terminal 50 generates the diagnosis information, the medication information, and the like related to the managed subject.

The storage unit 52 of the physician terminal 50 stores the diagnosis information, the medication information, and the like related to the managed subject generated by the control unit 51. Further, the storage unit 52 stores the management screen data and the like received from the server 70.

The communication unit 53 is a communication interface for performing communication with the server 70 (and the portable terminal 30). The communication unit 53 transmits the diagnosis information, the medication information, and the like related to the managed subject to the server 70. Further, the communication unit 53 can receive the management screen data and the like from the server 70.

The operation unit 54 and the display unit 55 are similar to the operation unit 14 and the display unit 15 of the blood pressure measurement device 10, respectively. At the operation unit 54, the medication information and the test information acquired by various test devices, and the like can be input. The medication information and the test information may be recorded in the server 70 via a network, such as a hospital internal LAN. Further, the display unit 55 can display the side effect management screen and the dosing adherence rate management screen on the basis of the management screen data generated by the medication management circuit 80. The display unit 55 is an example of a display screen.

### 2.2.4 Hardware Configuration Example of Server

Next, a hardware configuration example of the server 70 is described. FIG. 6 is a block diagram illustrating an example of the hardware configuration of the server 70 according to the present embodiment. As illustrated in FIG. 6, the server 70 according to the present embodiment includes a control unit 71, a storage unit 72, a communication unit 73, and the medication management circuit 80.

The control unit 71 and the storage unit 72 are similar to the control unit 11 and the storage unit 12 of the blood pressure measurement device 10, respectively. The storage unit 72 of the server 70 may store information transmitted from the portable terminal 30 and the physician terminal 50, the management screen data generated by the medication management circuit 80, and the like.

The communication unit 73 is a communication interface for performing communication with the portable terminal 30 and the physician terminal 50. For example, the communication unit 73 receives, from the portable terminal 30, the blood pressure information, the pulse information, the activity information, the environment information, and the like. The communication unit 73 receives, from the physician terminal 50, the test information, the diagnosis information, the medication information, and the like related to the managed subject. The communication unit 73 can transmit information stored in the storage unit 72 to the portable terminal 30 and the physician terminal 50.

The medication management circuit 80 includes a processor 80a and a memory 80b, for example. The medication management circuit 80 implements various operation controls, data processing, and the like by the processor 80a executing programs stored in the memory 80b. Further, the medication management circuit 80 can include a clock (not illustrated) to clock the current date/time.

The processor 80a is, for example, a CPU or a micro processing unit (MPU) including an arithmetic circuit, or the like. The processor 80a is capable of executing the control and data processing of each unit by executing a program stored in the memory 80b or the storage unit 72.

The memory 80b includes, for example, a non-volatile memory that stores programs to be executed by the processor 80a, and a volatile memory, such as a RAM, for use as a working memory.

The medication management circuit 80 executes the side effect management process on the basis of the side effect management program. The side effect management program is an example of a medication management program. The side effect management process by the medication management circuit 80 will be described later. The side effect management program is a program for causing the medication management circuit 80 to execute the side effect management process. The side effect management program may be stored in the memory 80b or stored in the storage unit 72.

Further, the medication management circuit 80 executes a dosing adherence rate management process on the basis of a dosing adherence rate management program. The dosing adherence rate management program is an example of a medication management program. The dosing adherence rate management process by the medication management circuit 80 will be described later. The dosing adherence rate management program is a program for causing the medication management circuit 80 to execute the dosing adherence rate management process. The dosing adherence rate management program may be stored in the memory 80b or may be stored in the storage unit 72.

Note that the control unit 71 may function as the medication management circuit 80. That is, the control unit 71 may also be used as the medication management circuit 80. In this case, the CPU of the control unit 71 serves as the processor 80a of the medication management circuit 80, the ROM of the control unit 71 serves as the non-volatile memory of the memory 80b of the medication management circuit 80, and the RAM of the control unit 71 serves as the volatile memory of the memory 80b of the medication management circuit 80.

### 2.3 Functional Configuration Examples

Next, an example of a functional configuration of the medication management system according to the present embodiment is described.

### 2.3.1 Functional Configuration Example of Medication Management Circuit

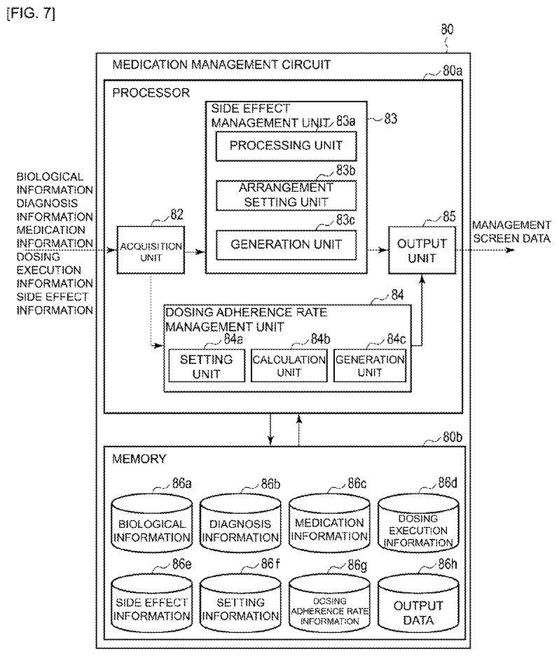

FIG. 7 is a block diagram schematically illustrating an example of a functional configuration of the medication management circuit 80 of the medication management system according to the present embodiment.

The processor 80a of the medication management circuit 80 deploys the medication management program stored in the non-volatile memory of the memory 80b in the volatile memory of the memory 80b. The processor 80a then construes and executes the medication management program deployed in the volatile memory, thereby functioning as an acquisition unit 82, a side effect management unit 83, a dosing rate management unit 84, and an output unit 85.

The volatile memory of the memory 80b functions as a biological information storage unit 86a, a diagnosis information storage unit 86b, a medication information storage unit 86c, a dosing execution information storage unit 86d, a side effect information storage unit 86e, a setting information storage unit 86f, a dosing adherence rate information storage unit 86g, and an output data storage unit 86h.

The biological information storage unit 86a stores the biological information related to each managed subject. The biological information is information related to a biological parameter of the managed subject. The biological information includes age, gender, blood pressure information, pulse information, and the like. The blood pressure information is an example of biological information.

The blood pressure information includes a blood pressure value. The blood pressure value is a systolic blood pressure, a diastolic blood pressure, or other index. The blood pressure information can also include a measured date/time and a measurement location for each blood pressure value. The pulse information includes a pulse value and a pulse wave. The pulse information can include a measured date/time and a measurement location for each of the pulse value and the pulse wave. The measured date/time includes, for example, a measured date, a measured time, and the like. The measured time is, for example, morning, evening, or the like. The measurement location is, for example, home, workplace, hospital, or the like.

The diagnosis information storage unit 86b stores the diagnosis information related to each of the managed subjects. The diagnosis information can include a hospital visit date and a diagnosis result. The diagnosis result can include a hospital name, information related to an irregular pulse wave, and the like. The information related to an irregular pulse wave is, for example, a diagnosis result based on the pulse information. The information related to an irregular pulse wave includes a detected date of the irregular pulse wave, and the like.

The medication information storage unit 86c stores the medication information related to each of the managed subjects. The medication information is information related to the specified medicine of the medication. The medication information is used as a portion of the dosing information. The dosing information is information related to the actual dosing of the managed subject. The medication information includes, for each of the specified medicines of the medication, a medication start date, a medication end date, a medicine name, a class name of the medicine, a prescribed dosing time, a prescribed dosage, and the like. The medication start date and the medication end date are, for example, consistent with the hospital visit dates. The prescribed dosing time is, for example, morning, evening, or the like. The prescribed dosing time and the prescribed dosage are determined in the medication.

The class name of the medicine is an example of a classification name of a medicine. The class name of the medicine is, for example, a class name of an antihypertensive agent prescribed in hypertension therapeutic guidelines. The class name is, for example, calcium channel blocker (CCB), angiotensin-converting enzyme (ACE), angiotensin receptor blocker (ARB), thiazide-based diuretic, beta blocker, or the like.

The dosing execution information storage unit 86d stores dosing execution information related to each of the managed subjects. The dosing execution information is used as a portion of the dosing information. The dosing execution information includes a medicine name and a dosing date/time for each of the medicines actually taken. The dosing date/time includes a dosing date, a dosing time, and the like. The dosing time is, for example, morning, evening, or the like. Further, the presence or absence of an actual dosing may be included.

The side effect information storage unit 86e stores the side effect information related to each of the managed subjects. The side effect information is information related to the onset of symptoms of a side effect. The side effect information includes symptom information. The side effect information also includes the onset date for each piece of symptom information. The symptom information is information related to the symptoms of a side effect. The symptom information includes feel good, dry cough, headache, dizziness, palpitation, hot flash, swelling, and the like.

The setting information storage unit 86f stores setting information. The setting information includes information related to a unit medication period. The unit medication period is an example of a unit medication period. The information related to the unit medication period includes a start date/time, an end date/time, and the like for each of a plurality of the unit medication periods.

The dosing adherence rate information storage unit 86g stores dosing adherence rate information related to each of the managed subjects. The dosing adherence rate information is information related to a dosing adherence rate calculated by the dosing adherence rate management unit 84. The dosing adherence rate information includes a dosing adherence rate for each of the plurality of unit medication periods. Further, the dosing adherence rate information includes a dosing adherence rate for each of the specified medicines. The dosing adherence rate is a ratio of the number of times a medicine was actually taken to the number of doses prescribed by the medication.

The output data storage unit 86h temporarily stores the management screen data output to an external display device or the like as output data. The management screen data include the side effect management screen data, the dosing adherence rate management screen data, and the like. The side effect management screen data are image data for displaying the side effect management screen on the display screen of the display device. The dosing adherence rate management screen data are image data for displaying the dosing adherence rate management screen on the display screen of the display device. The side effect management screen and the dosing adherence rate management screen will be described later.

Note that, while an example has been described in which the information of each managed subject is stored the respective information storage units, the present invention is not limited thereto. For example, a plurality of storage units may be provided for each managed subject.

The acquisition unit 82 acquires the management information related to each of the managed subjects. The acquisition unit 82 acquires the management information from the communication unit 73 or the storage unit 72, for example. The management information includes, for example, the biological information, the diagnosis information, the medication information, the dosing information, the side effect information, and the like. The acquisition unit 82 stores the management information thus acquired in the memory 80b.

The side effect management unit 83 executes the side effect management process, thereby generating the side effect management screen data related to each managed subject on the basis of the management information stored in the memory 80b. The side effect management process will be described later. The side effect management screen data are display data for displaying a side effect management screen on the display screen of the display device. The side effect management screen data are displayed on the display screen, and thus the diagnosis display data for each day are arranged side-by-side on the side effect management screen. One day is an example of a unit diagnosis period. The display device is, for example, the physician terminal 50, and the display screen is, for example, the display unit 55. The display device may be, for example, the display unit 35 of the portable terminal 30. The side effect management unit 83 stores the generated medication management screen data in the output data storage unit 86h of the memory 80b.

The side effect management unit 83 includes a processing unit 83a, an arrangement setting unit 83b, and a generation unit 83c. The processing unit 83a generates a plurality of the diagnosis display data on the basis of the management information stored in the memory 80b. The diagnosis display data are each arranged in predetermined positions on the side effect management screen. The diagnosis display data include date/time display data, blood pressure display data, pulse display data, pulse wave display data, hospital visit display data, dosing display data, side effect display data, and the like. The diagnosis display data are each stored in the output data storage unit 86h of the memory 80b.

The date/time display data indicate information related to the date/time. The date/time display data indicate, for example, the date for each day. The date/time display data may display, in addition to the date, information related to time for each day. The information related to time is, for example, morning, evening, or the like.

The blood pressure display data indicate information related to blood pressure. The blood pressure display data are generated on the basis of, for example, the blood pressure information stored in the biological information storage unit 86a. The blood pressure display data indicate, for example, a systolic blood pressure and a diastolic blood pressure in the morning, and a systolic blood pressure and a diastolic blood pressure in the evening, for each day. The systolic blood pressure and the diastolic blood pressure in the evening are each, for example, an average value of the measured values of one day. Further, on the day that the managed subject visits the hospital, the blood pressure display data can include the systolic blood pressure and the diastolic blood pressure measured at the hospital. The blood pressure display data constitute, for example, a graph showing fluctuations in blood pressure.

The pulse display data indicate information related to the pulse value. The pulse display data are generated on the basis of, for example, the pulse information stored in the biological information storage unit 86a. The pulse display data constitute, for example, a graph showing fluctuations in pulse value for each day. The pulse display data can also include morning and evening pulse values and pulse values measured at the hospital, similar to the blood pressure display data.

The pulse wave display data indicate information related to an irregular pulse wave. The pulse wave display data are generated on the basis of, for example, the pulse wave information stored in the biological information storage unit 86a. The pulse wave display data constitute, for example, an icon indicating that an irregular pulse wave has been detected.

The hospital visit display data indicate information related to a hospital visit. The hospital visit display data are generated on the basis of, for example, the diagnosis information stored in the diagnosis information storage unit 86b. The hospital visit display data constitute, for example, an icon indicating a hospital visit.

The dosing display data indicate information related to actual dosing. The dosing display data are generated on the basis of, for example, the dosing information stored in the dosing execution information storage unit 86d. The dosing display data indicate, for example, a medicine name, a dosing date/time, a dosage, and the like. The dosing display data include medication display data and dosing execution display data.

The medication display data indicate information related to a specified medication. The medication display data are generated on the basis of, for example, the medication information stored in the medication information storage unit 86c. The medication display data indicate, for example, a medicine name, a class name of the medicine, a prescribed dosing date/time, a prescribed dosage, and the like.

The dosing execution display data indicate information related to whether a medicine was actually taken on the basis of the specified medication. The dosing execution display data are generated, for example, on the basis of the dosing execution information stored in the dosing execution information storage unit 86d. The dosing execution display data constitute, for example, an icon indicating that a medicine was actually taken on the basis of the medication indicated in the medication display data.

The side effect display data indicate information related to side effects. The side effect display data are generated on the basis of, for example, the side effect information stored in the side effect information storage unit 86e. The side effect display data constitute, for example, an icon indicating the onset of symptoms of a side effect and the onset symptom.

The arrangement setting unit 83b sets arrangement information for each element included in the respective diagnostic display data on the basis of the setting information stored in the setting information storage unit 86f, and stores the arrangement information in the output data storage unit 86h of the memory 80b. The arrangement information is information used to determine a positional relationship between the diagnosis display data on the side effect management screen, and is information related to the corresponding date/time of each element of the respective diagnosis display data. The arrangement information is not displayed on the side effect management screen. For example, the arrangement setting unit 83b sets the measured date and measured time as the arrangement information for each element included in the blood pressure display data. Further, the arrangement setting unit 83b sets, as the arrangement information, the measured date of each element included in the pulse display data, the detected date of an irregular pulse wave of each element included in the pulse wave display data, the hospital visit date of each element included in the hospital visit display data, the prescribed date and the prescribed time of each element included in the medication display data, the dosing date and the dosing time of each element included in the dosing execution display data, and the onset date of each element included in the side effect display data.

The generation unit 83c generates the side effect management screen data on the basis of the diagnosis display data and the arrangement information. Specifically, the generation unit 83c arranges each element included in the respective diagnostic display data on the side effect management screen in association with each date. Thus, the side effect management screen data are displayed on the display screen, making it possible to display each element of the diagnosis display data of each day side-by-side. “One day” is an example of a unit diagnosis period.

The dosing adherence rate management unit 84 executes the dosing adherence rate management process, thereby calculating a dosing adherence rate for the managed subject on the basis of the medication information, the dosing information, and the unit medication period, and generates the dosing adherence rate management screen data. The dosing adherence rate management process will be described later. The dosing adherence rate management screen data are display data for displaying the dosing adherence rate management screen on the display screen of the display device. The dosing adherence rate management screen data are displayed on the display screen, thereby displaying the medication information and the dosing adherence rate for each unit medication period and for each medicine on the dosing adherence rate management screen. The display device is, for example, the physician terminal 50, and the display screen is, for example, the display unit 55. The dosing adherence rate management unit 84 stores the generated dosing adherence rate management screen data in the output data storage unit 86h of the memory 80b.

The dosing adherence rate management unit 84 includes a setting unit 84a, a calculation unit 84b, and a generation unit 84c, for example. The setting unit 84a sets at least one unit medication period on the basis of the medication information stored in the medication information storage unit 86c. The start date of the unit medication period is, for example, consistent with the start date of the medication, and the end date of the unit medication period is, for example, consistent with a medication end date. Further, the unit medication period is, for example, consistent with a period from a hospital visit date to the next hospital visit date. In a case where the next hospital visit date cannot be identified, the end date of the unit medication period may be, for example, the last day in the acquired dosing information. Alternatively, the end date of the unit medication period may be configured to be specifiable as desired by the administrator, for example. In each unit medication period, the medicine identification information (medicine name, for example), the prescribed dosing time, the prescribed dosage, and the like specified in the medication are the same. The unit medication period may be one day or may be a number of days. Further, lengths of the unit medication periods may differ from each other. The set unit medication period is stored in the setting information storage unit 86f of the memory 80b as setting information.

The calculation unit 84b calculates the dosing adherence rate for each unit medication period and for each medicine on the basis of the medication information, the dosing execution information, and the setting information. The dosing adherence rate of a specific medicine in a specific unit medication period is a ratio of the number of times that the specific medicine was actually taken to the prescribed number of doses of the specific medicine within the specific unit medication period. The calculated dosing adherence rate is stored in the dosing adherence rate information storage unit 86g of the memory 80b as the dosing adherence rate information.

Note that in the calculation of the dosing adherence rate in a specific unit medication period, the calculation unit 84b further calculates the dosing adherence rate for each prescribed dosing period in a case where a plurality of prescribed dosing times are prescribed in a single day of medication during a specific unit medication period. The dosing adherence rate at a specific prescribed dosing time is a ratio of the number of times the medicine was actually taken to the prescribed number of doses at a specific prescribed dosing time within a specific unit medication period.

The generation unit 84c generates the dosing adherence rate management screen data on the basis of the medication information and the dosing adherence rate information. The generation unit 84c arranges the medication information and the dosing adherence rate information in correspondence with each unit medication period on the dosing adherence rate management screen. The dosing adherence rate management screen data are displayed on the display screen, thereby displaying the medication information and the dosing adherence rate information for each unit medication period. The generated dosing adherence rate management screen data are stored in the output data storage unit 86h of the memory 80b.

The output unit 85 can output, to the physician terminal 50, the side effect management screen data generated by the side effect management unit 83 and stored in the output data storage unit 86h of the memory 80b, or the dosing adherence rate management screen data generated by the dosing adherence rate management unit 84 and stored in the output data storage unit 86h of the memory 80b, in accordance with an instruction from the side effect management unit 83 or the dosing adherence rate management unit 84.

### 2.4 Operation Examples

Next, an operation example of the medication management system according to the present embodiment is described. Note that the processing procedure described below is merely an example, and each process may be changed to the extent possible. Further, in the processing procedure described below, steps can be omitted, substituted, and added in accordance with the embodiment as appropriate.

### 2.4.1 Operation Example of Medication Management System

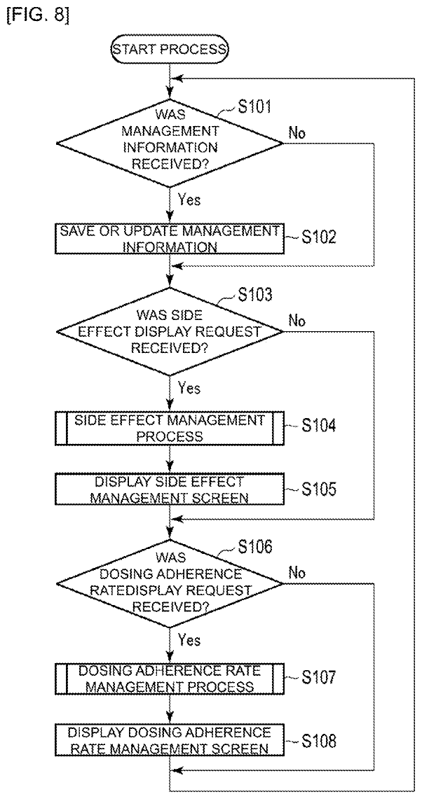

FIG. 8 is a flowchart illustrating an example of a processing procedure by the control unit 71 of the server 70 and the processor 80a of the medication management circuit 80 in medication management using the medication management system according to the present embodiment. The medication management process using the medication management system is executed by an administrator, such as, for example, a physician. In the medication management process, the processing of S101 to S108 described below is repeated.

In the medication management process, the control unit 71 of the server 70 determines whether or not new management information has been received (S101). For example, the management information is transmitted from the portable terminal 30 or the physician terminal 50, and received by the communication unit 73 of the server 70. In a case where new management information is received (S101—Yes), the control unit 71 stores the newly acquired management information in the storage unit 72 (S102). Thus, the information related to the managed subject is additionally saved or updated in an electronic medical chart, for example. In a case where new management information is not received (S101—No), the processing proceeds to S103.

In the processing of S103, the control unit 71 of the server 70 determines whether or not a side effect display request has been received. The side effect display request is information indicating that an instruction for displaying the side effect management screen on the display device has been input. The instruction for displaying the side effect management screen on the display device is input at the operation unit 54 of the physician terminal 50, for example. Then, information indicating that an instruction for displaying the side effect management screen on the display device, transmitted from the physician terminal 50, has been input is received by the communication unit 73 of the server 70. In a case where a side effect display request is not received (S103—No), the processing proceeds to S106.

In a case where a side effect display request is received (S103—Yes), the control unit 71 transmits information indicating that a side effect display request has been received to the processor 80a of the medication management circuit 80. The processor 80a executes the side effect management process on the basis of having acquired information indicating that a side effect display request has been received (S104). The side effect management process will be described later. The server 70 outputs the side effect management screen data output from the medication management circuit 80 in the side effect management process to the physician terminal 50 as the management screen data. The control unit 51 of the physician terminal 50 displays the side effect management screen on the display unit 55 on the basis of the received side effect management screen data (S105).

In the processing of S106, the control unit 71 of the server 70 determines whether or not a dosing adherence rate display request has been received. The dosing adherence rate display request is information indicating that an instruction for displaying the dosing adherence rate screen on the display device has been input. The instruction for displaying the dosing adherence rate screen on the display device is input at the operation unit 54 of the physician terminal 50, for example. Then, information indicating that an instruction for displaying the dosing adherence rate screen on the display device, transmitted from the physician terminal 50, has been input is received by the communication unit 73 of the server 70. In a case where a dosing adherence rate display request is not received (S106—No), the processing returns to S101.

In a case where a dosing adherence rate display request is received (S106—Yes), the control unit 71 transmits information indicating that a dosing adherence rate display request has been received to the processor 80a of the medication management circuit 80. The processor 80a executes the dosing adherence rate management process on the basis of having acquired information indicating that a dosing adherence rate display request has been received (S107). The dosing adherence rate management process will be described later. The server 70 outputs the dosing adherence rate management screen data output from the medication management circuit 80 in the dosing adherence rate management process to the physician terminal 50 as the management screen data. The control unit 51 of the physician terminal 50 displays the dosing adherence rate management screen on the display unit 55 on the basis of the received dosing adherence rate management screen data (S108).

### 2.4.2 Operation Example of Medication Management Circuit in Side Effect Management Process

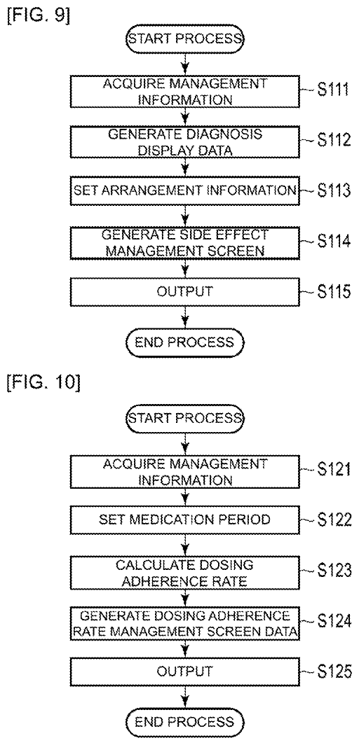

FIG. 9 is a flowchart illustrating an example of a procedure of the side effect management process in the processor 80a of the medication management circuit 80 according to the present embodiment. The processor 80a starts the side effect management process on the basis of, for example, an operation input performed at the physician terminal 50.

In the side effect management process, the processor 80a first acquires the management information related to the managed subject from the storage unit 72 of the server 70, for example (S111). The management information includes the biological information, the diagnosis information, the medication information, the dosing information, and the side effect information.

Next, the processor 80a generates the diagnosis display data on the basis of the management information (S112). The diagnosis display data include elements such as, for example, the date/time display data, the blood pressure display data, the pulse display data, the pulse wave display data, the hospital visit display data, the medication display data, the dosing execution display data, and the side effect display data.

Next, the processor 80a sets the arrangement information for each element of the respective diagnosis display data on the basis of the management information (S113).

Next, the processor 80a generates the side effect management screen data on the basis of the diagnostic display data and the arrangement information (S114). Thus, the processor 80a arranges each element of the respective diagnosis display data in correspondence with each date on the side effect management screen.

Next, the processor 80a outputs the side effect management screen data to the outside as the management screen data (S115). The processor 80a outputs the side effect management screen data to, for example, the physician terminal 50. The processor 80a may output the side effect management screen data to the portable terminal 30.

### 2.4.3 Operation Example of Medication Management Circuit in Dosing Adherence Rate Management Process

FIG. 10 is a flowchart illustrating an example of the procedure of the dosing adherence rate management process in the processor 80a of the medication management circuit 80 according to the present embodiment. The processor 80a starts the dosing adherence rate management process on the basis of, for example, an operation input performed at the physician terminal 50.

In the dosing adherence rate management process, the processor 80a first acquires the management information related to the managed subject from the storage unit 72 of the server 70, for example (S121). The management information includes medication information and dosing information.

Next, the processor 80a sets the unit medication period (S122). For example, the processor 80a sets a plurality of the unit medication periods on the basis of hospital visit information.

Next, the processor 80a calculates the dosing adherence rate for each unit medication period and for each medicine on the basis of the medication information, the dosing information, and the setting information of the unit medication period (S123). The processor 80a calculates the dosing adherence rate in the unit medication period for all set unit medication periods as well as for all specified medicines. In the calculation of the dosing adherence rate of a specific medicine in a specific unit medication period, the processor 80a first calculates, in the calculation unit 84b, the prescribed number of doses within a specific unit medication period on the basis of the medication start date and the medication end date included in the medication information. Next, the processor 80a calculates the actual number of doses of the specific medicine within the specific unit medication period on the basis of the dosing date/time included in the dosing information for the specific medicine. Then, the processor 80a calculates the ratio of the actual number of doses to the prescribed number of doses as the dosing adherence rate for the specific medicine in the specific unit medication period.

Next, the processor 80a generates the dosing adherence rate management screen data on the basis of the medication information and the dosing adherence rate information (S124). Thus, the processor 80a arranges the medication information and the dosing adherence rate information in correspondence with each unit medication period and in correspondence with each medicine on the dosing adherence rate management screen.

Next, the processor 80a outputs the dosing adherence rate management screen data to the outside as the management screen data (S125). The processor 80a outputs the dosing adherence rate management screen data to the physician terminal 50, for example.

### 2.5 Actions and Effects

Next, actions and effects of the medication management system according to the present embodiment are described.

### 2.5.1 Display Example of Side Effect Management Screen

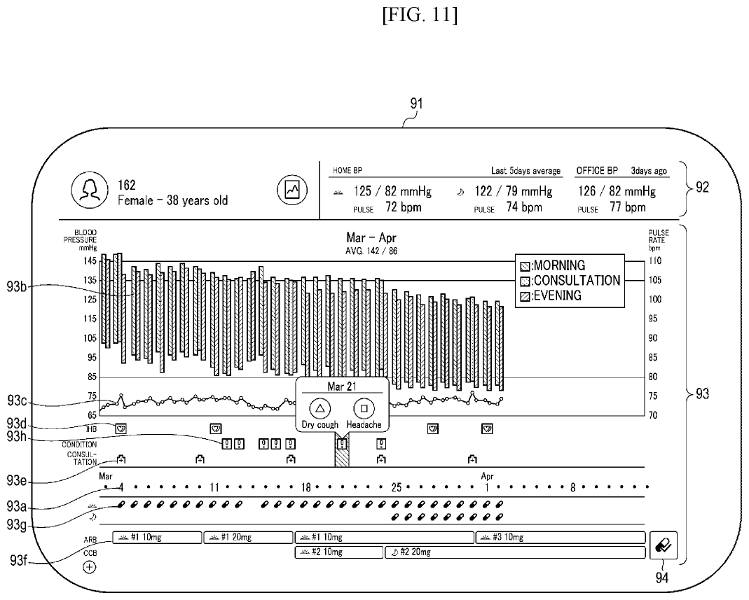

FIG. 11 illustrates a display example of the side effect management screen generated by the side effect management process of the medication management circuit 80. A side effect management screen 91 is an example of the side effect management screen. The side effect management screen 91 is, for example, displayed on the display unit 55 of the physician terminal 50, and is used for diagnosis related to the managed subject by the administrator. The managed subject is, for example, a hypertension patient. The administrator is, for example, a physician. The side effect management screen 91 may be displayed on, for example, the display unit 35 of the portable terminal 30 of the managed subject.

The side effect management screen 91 includes a representative information display portion 92, a diagnosis information display portion 93, and a display switching portion 94. In the representative information display portion 92, personal information and representative information are displayed. The personal information is information for identifying an individual as the managed subject. The personal information includes a management number, a gender, an age, and the like. The personal information may further include a name and the like. The representative information includes representative values such as, for example, those for systolic blood pressure, diastolic blood pressure, and pulse over several recent days.

In the diagnosis information display portion 93, the diagnosis display data are arranged. The diagnosis information display portion 93 includes a date display portion 93a, a blood pressure display portion 93b, a pulse display portion 93c, a pulse wave display portion 93d, a hospital visit display portion 93e, a medication display portion 93f, a dosing execution display portion 93g, and a side effect display portion 93h.

In the date display portion 93a, the date/time display data are displayed. In the date display portion 93a, the date is displayed for each day as the date/time display data. “One day” is an example of a unit diagnosis period.

In the blood pressure display portion 93b, the blood pressure display data are displayed. In the blood pressure display portion 93b, a blood pressure graph indicating fluctuations in blood pressure is displayed as the blood pressure display data. The blood pressure graph is a vertical bar graph. In the blood pressure graph, blood pressure value (BLOOD PRESSURE) is used for the vertical axis, and the dates of the date display portion 93a are used for the horizontal axis. The blood pressure graph includes a plurality of measured values as elements. The measured values are those of the systolic blood pressure and the diastolic blood pressure measured at home, those of the systolic blood pressure and the diastolic blood pressure measured at hospital visits, and the like. Further, the blood pressure graph is a vertical bar graph with systolic blood pressure as the upper end and diastolic blood pressure as the lower end. The blood pressure display portion 93b is arranged with the measured dates of each measured value corresponding to the dates of the date display portion 93a. Further, the respective measured values are classified into a measured value of the morning and a measured value of the evening, and displayed accordingly.

In the pulse display portion 93c, the pulse display data are displayed. In the pulse display portion 93c, a pulse graph indicating fluctuations in pulse value is displayed as the pulse display data. The pulse graph is a line graph. In the pulse graph, pulse value (pulse rate) is used for the vertical axis, and the dates of the date display portion 93a are used for the horizontal axis. The pulse graph includes a plurality of measured values as elements. The pulse display portion 93c is arranged with the measured dates of each measured value corresponding to the dates of the date display portion 93a.

In the pulse wave display portion 93d, the pulse wave display data are displayed. In the pulse wave display portion 93d, icons indicating that an irregular heartbeat (IHB) has been detected are displayed as elements of the pulse wave display data. The pulse wave display portion 93d is arranged with the detected dates of each icon corresponding to the dates of the date display portion 93a.

In the hospital visit display portion 93e, the hospital visit display data are displayed. In the hospital visit display portion 93e, icons indicating that a hospital visit was made are displayed as elements of the hospital visit display data. The hospital visit display portion 93e is arranged with the hospital visit dates of each icon corresponding to the dates of the date display portion 93a.

In the medication display portion 93f, the medication display data are displayed. In the medication display portion 93f, a medicine name, an icon indicating a prescribed dosing time, and a prescribed dosage are displayed for each medicine as the medication display data. The icon indicating a prescribed dosing time shows, for example, a picture of a rising sun, indicating that the prescribed dosing time is morning. Or, the icon indicating a prescribed dosing time shows, for example, a picture of a crescent moon, indicating that the prescribed dosing time is evening. In the medication display portion 93f, medicines #1 to #3 are each displayed as medicine names. Medicines #1 to #3 are hypertension therapeutic agents. Further, hypertension therapeutic agents are one example of therapeutic agents for diseases requiring home management. Here, medicine #1 and medicine #3 are classified as class 1 and medicine #2 is classified as class 2. The respective medication display data are classified into classes of medicine and displayed accordingly. The medication display portion 93f is arranged with the prescribed dosing dates of the respective medication display data corresponding to the dates of the date display portion 93a.

In the dosing execution display portion 93g, the dosing execution display data are displayed. In the dosing execution display portion 93g, icons indicating that medicine was taken are displayed as elements of the dosing execution display data. The dosing execution display portion 93g is arranged with the dosing dates of each icon corresponding to the dates of the date display portion 93a. Each icon is classified into morning dosing and evening dosing, and displayed accordingly. Each icon indicates that, on the date corresponding to the date display portion 93a, medicine was taken on the basis of the medication displayed in the medication display portion 93f. That is, each icon of the dosing execution display portion 93g is used in combination with the display of the medication display portion 93f, thereby indicating the name of the medicine taken, the class name of the medicine taken, the actual dosing date/time, the actual dosage, and the like.

In the side effect display portion 93h, the side effect display data are displayed. In the side effect display portion 93h, icons indicating the onset of symptoms of a side effect are displayed as elements of the side effect display data. The side effect display portion 93h is arranged with the onset dates of each icon corresponding to the dates of the date display portion 93a. In the side effect display portion 93h, selecting an icon displays an icon indicating the symptoms of the side effect. The icons indicating the symptoms of a side effect each indicate any one of dry cough, headache, dizziness, palpitation, hot flash, swelling, or the like. For example, in the one example in FIG. 11, it is indicated that, on March 21, the symptoms of dry cough and headache developed.

In the display switching portion 94, an icon configured to switch the display from the side effect management screen to the dosing adherence rate management screen upon selection is displayed. For example, selecting the icon of the display switching portion 94 by operating the operation unit 54 of the physician terminal 50 causes an operation for displaying the dosing adherence rate management screen to be input.

### 2.5.2 Diagnosis Example Based on Side Effect Management Screen

In the one example in FIG. 11, the management information from March 3 to April 2 is reflected on the side effect management screen 91. Here, a period from March 4 to March 10 is a first diagnosis period P1. Further, a period from March 10 to March 17 is a second diagnosis period P2, a period from March 17 to March 24 is a third diagnosis period P3, a period from March 24 to March 31 is a fourth diagnosis period P4, and a period from March 31 to the next hospital visit date is a fifth diagnosis period P5. In each of the diagnosis periods P1 to P5, the start date and the end date coincide with hospital visit dates. Thus, each of the diagnosis periods P1 to P5 is a period from a hospital visit date to the next hospital visit date. The start dates and the end dates of the diagnosis periods P1 to P5 coincide with medication change dates. Each of the diagnosis periods P1 to P5 is an example of a unit diagnosis period. The unit diagnosis period may be a period from a day following a hospital visit date to the next hospital visit date, or may be one day.

The administrator acquires, on the basis of the displays of the date display portion 93a, the medication display portion 93f, and the dosing execution display portion 93g, the names of the medicines actually taken by the managed subject and the actual dosages for each of the diagnosis periods P1 to P5.

For example, in the first diagnosis period P1, as illustrated in the medication display portion 93f, medication #1 is specified. Further, in the medication of the first diagnosis period P1, the prescribed dosage of medicine #1 is 10 mg, and the prescribed dosing time of medicine #1 is morning. The administrator determines that, for the dates and the times displayed by the icons in the dosing execution display portion 93g, the managed subject has taken the prescribed dosages of the medicines specified in the medication. Here, as illustrated in the dosing execution display portion 93g, an icon indicating that a morning-prescribed medicine was taken is displayed for all dates in the first diagnosis period P1. Accordingly, the administrator determines that the managed subject has taken 10 mg of medicine #1 in the morning in the first diagnosis period P1.

Further, the administrator determines, on the basis of the displays of the date display portion 93a and the blood pressure display portion 93b, whether the blood pressure of the managed subject exceeds a hypertension reference value for each of the diagnosis periods P1 to P5. Blood pressure is an example of biological information.

For example, in the first diagnosis period P1, as indicated in the blood pressure display portion 93b, the systolic blood pressure is greater than 135 mmHg and the diastolic blood pressure is greater than 85 mmHg. Here, 135 mmHg is the reference value for hypertension in relation to systolic blood pressure. Further, 85 mmHg is the reference value for hypertension in relation to diastolic blood pressure. Accordingly, the administrator determines that the systolic blood pressure and the diastolic blood pressure exceed the reference values for hypertension in the first diagnosis period P1.

Further, the administrator acquires, on the basis of the displays of the date display portion 93a and the side effect display portion 93b, the onset status of side effects in the managed subject for each of the diagnosis periods P1 to P5.

For example, as indicated in the side effect display portion 93h, in the second diagnosis period P2, many icons indicating the onset of side effects are displayed. Accordingly, the administrator determines that side effects have frequently occurred in the managed subject in the second diagnosis period P2.

The administrator acquires the information required to determine an effective medication and an effective hypertension agent for the managed subject on the basis of the displays of the date display portion 93a, the medication display portion 93f, the dosing execution display portion 93g, and the side effect display portion 93h.

For example, in the second diagnosis period P2, the systolic blood pressure and the diastolic blood pressure are substantially the same as the respective reference values for hypertension, and side effects frequently occurred. Therefore, the administrator determines that the medication in the second diagnosis period P2 is not an appropriate medication for the managed subject. Accordingly, the administrator determines that medicine #1 is not an appropriate medicine for the managed subject.

In the mornings of the third diagnosis period P3, the systolic blood pressure and the diastolic blood pressure are substantially the same as the respective reference values for hypertension. Further, in the evenings of the third diagnosis period P3, the systolic blood pressure and the diastolic blood pressure are less than the respective reference values for hypertension. Further, in the third diagnosis period P3, no side effects occurred. Accordingly, the administrator determines that the medication of the third diagnosis period P3 is a more appropriate medication for the managed subject than the medication of the first diagnosis period P1 and the medication of the second diagnosis period P2.

Here, in the third diagnosis period P3, as compared to the second diagnosis period P2, a portion of the total dosage is changed from medicine #1 to medicine #2. Accordingly, the administrator determines that medicine #2 is a more appropriate medicine than medicine #1 for the managed subject. Further, medicine #1 is classified as class 1 and medicine #2 is classified as class 2. Accordingly, the administrator determines that the medicine classified as class 2 is a more appropriate medicine than the medicine classified as class 1 for the managed subject.

In the mornings of the fourth diagnosis period P4, the systolic blood pressure and the diastolic blood pressure are less than the respective reference values for hypertension. Further, in the evenings of the fourth diagnosis period P4, the systolic blood pressure and the diastolic blood pressure are less than the respective reference values for hypertension.

Further, in the fourth diagnosis period P4, almost no side effects occurred. Accordingly, the administrator determines that the medication of the fourth diagnosis period P4 is a more appropriate medication for the managed subject than the medications of the first diagnosis period P1 to the third diagnosis period P3.

### 2.5.3 Display Example of Dosing Adherence Rate Management Screen

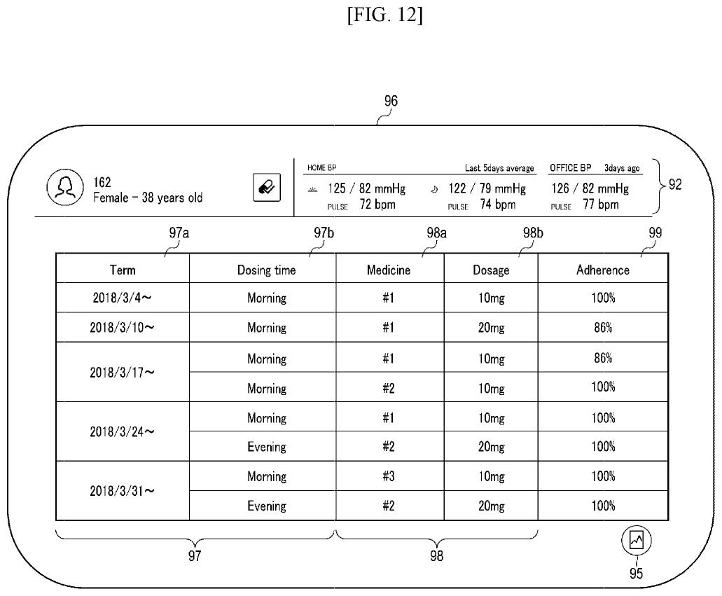

FIG. 12 illustrates a display example of the dosing adherence rate management screen generated by the dosing adherence rate management process of the medication management circuit 80. The dosing adherence rate management screen 96 displays the dosing adherence rate of the managed subject for each set unit medication period. The dosing adherence rate management screen 96 is an example of a dosing adherence rate management screen. The dosing adherence rate management screen 96 is displayed on the display unit 55 of the physician terminal 50, for example, and is used for diagnosis of the managed subject by the administrator. The managed subject is, for example, a hypertension patient. The administrator is, for example, a physician.

The dosing adherence rate management screen 96 includes the representative information display portion 92, a display switching portion 95, a period information display portion 97, a medication information display portion 98, and a dosing adherence rate display portion 99. In the representative information display unit 92, the personal information and the representative information are displayed. The personal information is information for identifying an individual as the managed subject. The personal information includes a management number, a gender, an age, and the like. The personal information may further include a name and the like. The representative information includes representative values such as, for example, those for systolic blood pressure, diastolic blood pressure, and pulse over several recent days.

In the display switching portion 95, an icon configured to switch the display from the dosing adherence rate management screen to the side effect management screen upon selection is displayed. For example, selecting the icon of the display switching portion 95 by operating the operation unit 54 of the physician terminal 50 causes an operation for displaying the side effect management screen to be input.

In the period information display portion 97, information related to the unit medication period is displayed. The period information display portion 97 includes a period display portion 97a and a time display portion 97b. In the period display portion 97a, for example, the start date of each of the unit medication periods is displayed.

In the time display portion 97b, information related to the prescribed dosing time (Dosing Time) is displayed for each unit medication period. In the time display portion 97b, for example, either “Morning” or “Evening” is displayed. When “Morning” is displayed in the time display portion 97b, the display indicates that the prescribed dosing time is morning. When “Evening” is displayed in the time display portion 97b, the display indicates that the prescribed dosing time is evening.

In the medication information display portion 98, the medication information for each unit medication period is displayed. The medication information displayed in the medication information display portion 98 includes, for example, the medicine identification information, the prescribed dosage, and the like. The medication information display portion 98 includes a medicine identification information display portion 98a and a dosage display portion 98b.

In the medicine identification information display portion 98a, the medicine name (Medicine) is displayed as the medicine identification information for each prescribed dosing time of the unit medication period. In the example illustrated in FIG. 12, any of medicines #1 to #3 is displayed as a medicine name in the medicine identification information display portion 98a. Medicines #1 to #3 are, for example, medicine names of hypertension therapeutic agents. Further, hypertension therapeutic agents are one example of a therapeutic agent for a disease requiring home management.

In the dosage display portion 98b, a prescribed dosage (Dosage) for each prescribed dosing time of the unit medication period and for each medicine is displayed.

In the dosing adherence rate display portion 99, a dosing adherence rate (Adherence) for each prescribed dosing time of the unit medication period and for each medicine is displayed.

### 2.5.4 Diagnosis Example Based on Dosing Adherence Rate Display Screen

In one example of FIG. 12, medication periods T1 to T5 are set. The respective start dates for each of the medication periods T1 to T5 coincide with the medication start dates. The respective end dates for each of the medication periods T1 to T5 coincide with the end dates of medication. The end date for medication period T5 is, for example, the final acquisition date of the dosing information. Further, in each of the medication periods T1 to T5, the start date and the end date coincide with hospital visit dates. The start dates and the end dates of the medication periods T1 to T5 coincide with medication change dates. The start date of the medication period T5 also coincides with a hospital visit date. Each of the medication periods T1 to T5 is an example of a unit medication period. In the present embodiment, the respective medication periods T1 to T5 coincide with the diagnosis periods P1 to P5. That is, the first medication period T1 is the period from March 4 to March 10. Further, the second medication period T2 is the period from March 10 to March 17. The third medication period T3 is the period from March 17 to March 24. The fourth medication period T4 is the period from March 24 to March 31. The fifth medication period T5 is the period from March 31 to April 2, which is the final acquisition date of the dosing information.

Further, as indicated in the medication information display portion 98, a plurality of prescribed dosing periods are set for each of the fourth medication period T4 and the fifth medication period T5. In each of the fourth medication period T4 and the fifth medication period T5, morning (Morning) is set as the first prescribed dosing time, and evening (Evening) is set as the second prescribed dosing time.

The administrator assesses, on the basis of the displays of the medication information display portion 98 and the dosing adherence rate display portion 99, the impact of the medicines on adherence (enthusiasm for treatment) of the managed subject.

For example, the dosing adherence rate in the first medication period T1 is 100% and the dosing adherence rate in the second medication period T2 is 86%. Accordingly, the administrator determines that the medication of the second medication period T2 is a medication that lowers the dosing adherence rate of the managed subject more than the medication of the first medication period T1. Thus, the administrator determines that the medication of the second medication period T2 lowers the adherence (enthusiasm for treatment) of the managed subject more than the medication of the first medication period T1.

In this example, the medication in the second medication period T2 is the same medicine #1, but the prescribed dosage is increased compared to that of the medication in the first medication period T1. Accordingly, the administrator can determine that the increase in the dosage of medicine #1 is the cause of the lowering of the dosing adherence rate of the managed subject.

Thus, the administrator makes a determination that, in the medication in the third medication period T3, the prescribed dosage of medicine #1 is to be returned to the same dosage as that of the first medication period T1 and a different medicine #2 is to be added.

In the third medication period T3, the dosing adherence rate of medicine #1 is 86% and the dosing adherence rate of medicine #2 is 100%. Accordingly, the administrator determines that medicine #2 is a medicine that does not lower the dosing adherence rate of the managed subject more than medicine #1. Thus, the administrator determines that medicine #2 does not lower the adherence (enthusiasm for treatment) of the managed subject more than medicine #1. The administrator can determine that medicine #2 is a medicine that does not reduce the dosing adherence rate of the managed person, is easy to take, and is less likely to be missed.

### 2.6 Effect

In the present embodiment, in the treatment of a hypertension disorder, for example, the side effect management screen that displays, for each day, each set of information including the blood pressure information, the dosing information, and the side effect information related to the managed subject can be displayed on a display screen of a display device utilized by the administrator. By comparing the information on the side effect management screen for each day, the administrator can easily determine the medication that sufficiently exhibits a medicinal effect and has minimal side effects. Therefore, according to the present embodiment, in the treatment of a disease that requires medication management at home, such as a hypertension disorder, an administrator such as a physician can easily determine the efficacy and side effects of the medication for a managed subject, such as a patient.

Further, in the present embodiment, in the treatment of a hypertension disorder, for example, the dosing adherence rate management screen that displays each set of information, including the medication information and the dosing adherence rate information related to the managed subject for each unit medication period and for each medicine can be displayed on a display screen of a display device utilized by the administrator. By comparing the information on the dosing adherence rate management screen for each unit medication period and each medicine, the administrator can easily determine the medicine that does not lower adherence for the managed subject. Therefore, according to the present embodiment, in the treatment of a disease that requires medication management at home, such as a hypertension disorder, an administrator such as a physician can easily determine the impact on adherence (enthusiasm for treatment) of a managed subject, such as a patient, for each medicine.

Further, by switching between and utilizing the side effect management screen and the dosing adherence rate management screen, the administrator can easily determine the medication that sufficiently exhibits a medicinal effect, has minimal side effects, and does not lower adherence for the managed subject. Note that the side effect management screen and the dosing adherence rate management screen may be displayed side-by-side rather than displayed by switching between the two.

For example, in the one example of FIG. 11 and FIG. 12, as described above, using the dosing adherence rate management screen 96, it is understood that the dosing adherence rate lowered due to an increase in dosage in the second medication period T2. Further, using the side effect management screen 91, it is understood that side effects occurred frequently in the managed subject in the second diagnosis period P2 corresponding to the second medication period T2. Accordingly, the administrator can determine, by using both the side effect management screen 91 and the dosing adherence rate management screen 96, that the increase in dosage and the frequent occurrence of side effects in the period corresponding to the second medication period T2 and the second diagnosis period P2 are the causes of the lowered dosing adherence rate of the managed subject.

Further, the side effect management unit 83 can also further display the dosing adherence rate for each unit medication period on the side effect management screen. In this case, the side effect management unit 83 acquires the dosing adherence rate information from the dosing adherence rate information storage unit 86g, and generates the dosing adherence rate display data on the basis of the acquired dosing adherence rate information. As the dosing adherence rate display data, a graph showing fluctuations in the dosing adherence rate for each unit medication period is generated, for example. Then, the side effect management unit 83 arranges the dosing adherence rate display data on the side effect management screen, with the unit medication periods corresponding to the unit diagnosis periods, such as dates.

Note that, on the dosing adherence rate management screen generated by the dosing adherence rate management unit 84, the occurrence frequency of side effects and the like may be displayed for each unit diagnosis period.

### 2.7 Modified Examples

While the medication management device of the present embodiment has been described using as an example medication management in which a patient having a hypertension disorder is the managed subject, the present invention is not limited thereto. The medication management device may be used for medication management in which a patient having a lifestyle-related disease other than a hypertension disorder is the managed subject, for example. In this case, for example, a blood glucose level and a cholesterol level are used as the biological information and displayed on the side effect management screen.

Further, the medication management device may also be used for medication management in which a patient having a disease requiring medication management at home, such as asthma, is the managed subject, for example. In this case, for example, information related to the symptoms of asthma and the like are used as the biological information and displayed on the side effect management screen.

### 3. Common Configurations of Embodiments and the Like

A medication management device (1:80) includes an acquisition unit (2:82) configured to acquire medication information by medicine and dosing information by medicine related to a managed subject, a setting unit (3:84a) configured to set at least one unit medication period, a calculation unit (4:84b) configured to calculate a dosing adherence rate by medicine for each of the at least one unit medication periods on the basis of the medication information by medicine and the dosing information by medicine thus acquired and the at least one unit medication period thus set, and a generation unit (5:84c) configured to generate management screen data configured to display, in association with each other, the medication information by medicine thus acquired and the dosing adherence rate by medicine thus calculated for each of the at least one unit medication periods.

Note that the present invention is not limited to the embodiment, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### Supplementary Notes

A part or the entirety of the embodiment can be described as described in the following supplementary notes in addition to the scope of the claims, but the present invention is not limited thereto.

### Supplementary Note 1

A medication management device including

a hardware processor and
a memory,
the hardware processing being configured to
acquire medication information by medicine and dosing information by medicine related to a managed subject, and store the medication information by medicine and the dosing information by medicine thus acquired in the memory,
set at least one unit medication period, and store the at least one unit medication period thus set in the memory,
calculate a dosing adherence rate by medicine for each of the at least one unit medication periods on the basis of the medication information by medicine, the dosing information by medicine, and the at least one unit medication period thus stored in the memory, and
generate management screen data configured to display, in association with each other, the medication information by medicine and the dosing adherence rate by medicine thus stored in the memory, for each of the at least one unit medication periods.

### Supplementary Note 2

A medication management method executed by a device including a hardware processor and a memory, the method including

acquiring medication information by medicine and dosing information by medicine related to a managed subject, and storing the medication information by medicine and the dosing information by medicine thus acquired in the memory, by the hardware processor,
setting at least one unit medication period, and storing the at least one unit medication period thus set in the memory, by the hardware processor,
calculating a dosing adherence rate by medicine for each of the at least one unit medication periods on the basis of the medication information by medicine, the dosing information by medicine, and the at least one unit medication period thus stored in the memory, and storing the dosing adherence rate by medicine thus calculated in the memory, by the hardware processor, and
generating management screen data configured to display, in association with each other, the medication information by medicine and the dosing adherence rate by medicine thus stored in the memory, for each of the at least one unit medication periods, by the hardware processor.

### REFERENCE SIGNS LIST

1 Medication management device

2 Acquisition unit

3 Setting unit

4 Calculation unit

5 Generation unit

6 Output unit

10 Blood pressure measurement device

11 Control unit

12 Storage unit

13 Communication unit

14 Operation unit

15 Display unit

16 Blood pressure sensor

17 Acceleration sensor

18 Temperature/humidity sensor

30 Portable terminal

31 Control unit

32 Storage unit

33 Communication unit

34 Operation unit

35 Display unit

36 GPS receiver

50 Physician terminal

51 Control unit

52 Storage unit

53 Communication unit

54 Operation unit

55 Display unit

70 Server

71 Control unit

72 Storage unit

73 Communication unit

80 Medication management circuit

80a Processor

80b Memory

82 Acquisition unit

83 Side effect management unit

83a Processing unit

83b Arrangement setting unit

83c Generation unit

84 Dosing adherence rate management unit

84a Setting unit

84b Calculation unit

84c Generation unit

85 Output unit

86a Biological information storage unit

86b Diagnosis information storage unit

86c Medication information storage unit

86d Dosing execution information storage unit

86e Side effect information storage unit

86f Setting information storage unit

86g Dosing adherence rate information storage unit

86h Output data storage unit

91 Side effect management screen

92 Representative information display portion

93 Diagnosis information display portion

93a Date display portion

93b Blood pressure display portion

93c Pulse display portion

93d Pulse wave display portion

93e Hospital visit display portion

93f Medication display portion

93g Dosing execution display portion

93h Side effect display portion

94, 95 Display switching portion

96 Dosing adherence rate management screen

97 Period information display portion

97a Period display portion

97b Time display portion

98 Medication information display portion

98a Medicine identification information display portion

98b Dosage display portion

99 Dosing adherence rate display portion

P1 to P5 Diagnosis period

T1 to T5 Medication period

1. A medication management device comprising:
a processor configured to:
acquire medication information by medicine and dosing information by medicine related to a managed subject;
set a plurality of unit medication periods;
calculate a dosing adherence rate by medicine for each of the plurality of unit medication periods on the basis of the medication information by medicine and the dosing information by medicine thus acquired and the plurality of unit medication periods thus set; and
generate management screen data configured to display, in association with each other, the medication information by medicine thus acquired and the dosing adherence rate by medicine thus calculated for each of the plurality of unit medication periods,
the medication information including medicine identification information and a prescribed dosage by medicine, with at least one of the medicine identification information and the prescribed dosage by medicine differing for each of the plurality of unit medication periods.

2. The medication management device according to claim 1, wherein
the medication management device is configured to manage medication in a treatment for a managed subject having a hypertension disorder.

3. The medication management device according to claim 1, wherein
the processor is further configured to acquire, as the medication information, the medicine identification information, the prescribed dosage by medicine, and a medication start date and a medication end date by medicine.

4. The medication management device according to claim 1, wherein
the processor is further configured to acquire, as the dosing information, at least the medicine identification information and a dosing date by medicine.

5. The medication management device according to claim 4, wherein
the processor is further configured to determine, by medicine, whether the managed subject has taken the medicine on the basis of the dosing information.

6. The medication management device according to claim 1, wherein
the processor is further configured to:
further acquire a medication start date and a medication end date, and
set the medication start date as a start date of an unit medication period of the plurality of unit medication periods, and set the medication end date as an end date of the unit medication period.

7. The medication management device according to claim 6, wherein
the processor is further configured to acquire a first hospital visit date as the medication start date, and acquire a second hospital visit date corresponding to a hospital visit date following the first hospital visit date as the medication end date.

8. The medication management device according to claim 1, wherein
the processor is further configured to calculate, as the dosing adherence rate by medicine, a ratio of an actual number of doses to a prescribed number of doses by the medicine in the plurality of unit medication periods.

9. The medication management device according to claim 1, wherein
the processor is further configured to:
acquire, as the dosing information, at least a dosing date and a dosing time by medicine, and
set a plurality of prescribed dosing times in the plurality of unit medication periods and generate, on the basis of the dosing date and the dosing time by medicine thus acquired, management screen data configured to display, in association with each other, the medication information by medicine and the dosing adherence rate by medicine for each of the plurality of prescribed dosing times.

10. A non-transitory computer-readable storage medium storing a medication management program for causing a processor to execute functions in the medication management device described in claim 1.

11. A medication management method comprising:
acquiring medication information by medicine and dosing information by medicine related to a managed subject;
setting a plurality of unit medication periods;
calculating a dosing adherence rate by medicine for each of the plurality of unit medication periods on the basis of the medication information by medicine and the dosing information by medicine thus acquired and the plurality of unit medication periods thus set; and
generating management screen data configured to display, in association with each other, the medication information by medicine thus acquired and the dosing adherence rate by medicine thus calculated for each of the plurality of unit medication periods,
the medication information including medicine identification information and a prescribed dosage by medicine, with at least one of the medicine identification information and the prescribed dosage by medicine differing for each of the plurality of unit medication periods.

12. A non-transitory computer-readable storage medium storing a medication management program for causing a processor to execute functions in the medication management device described in claim 2.

13. A non-transitory computer-readable storage medium storing a medication management program for causing a processor to execute functions in the medication management device described in claim 3.

14. A non-transitory computer-readable storage medium storing a medication management program for causing a processor to execute functions in the medication management device described in claim 4.

15. A non-transitory computer-readable storage medium storing a medication management program for causing a processor to execute functions in the medication management device described in claim 5.

16. A non-transitory computer-readable storage medium storing a medication management program for causing a processor to execute functions in the medication management device described in claim 6.

17. A non-transitory computer-readable storage medium storing a medication management program for causing a processor to execute functions in the medication management device described in claim 7.

18. A non-transitory computer-readable storage medium storing a medication management program for causing a processor to execute functions in the medication management device described in claim 8.

19. A non-transitory computer-readable storage medium storing a medication management program for causing a processor to execute functions in the medication management device described in claim 9.
